(19) United States
(12) Patent Application Publication
(10) Pub. No.: US 2023/0187069 A1
(43) Pub. Date: Jun. 15, 2023

(54) ARTIFICIAL INTELLIGENCE APPARATUS FOR PLANNING AND EXPLORING OPTIMIZED TREATMENT PATH AND OPERATION METHOD THEREOF

(71) Applicant: ELECTRONICS AND TELECOMMUNICATIONS RESEARCH INSTITUTE, Daejeon (KR)

(72) Inventors: Jae Hun CHOI, Daejeon (KR); Do Hyeun KIM, Daejeon (KR); Hwin Dol PARK, Daejeon (KR)

(21) Appl. No.: 17/938,012

(22) Filed: Oct. 4, 2022

(30) Foreign Application Priority Data
Dec. 15, 2021 (KR) 10-2021-0179326
Jul. 14, 2022 (KR) 10-2022-0086864

(51) Int. Cl. G16H 50/20; G16H 50/70; G16H 10/60

(52) U.S. Cl. CPC G16H 50/20; G16H 50/70; G16H 10/60

(57) ABSTRACT

Disclosed is an artificial intelligence apparatus, which includes an episode conversion module that receives an electronic medical record (EMR) of a patient and converts the received EMR into an episode including a condition of the patient, a treatment method, and a treatment history, a patient condition predictive intelligence deep learning module that trains a patient condition predictive intelligence for predicting a following condition of the patient after applying the treatment method, a local policy intelligence reinforcement learning module that performs reinforcement learning of a policy intelligence for planning an optimized treatment path for the patient based on the episode, an optimized treatment path exploration module that plans the optimized treatment path for the patient by using the policy intelligence, and a global policy intelligence management module that updates a global policy intelligence for planning and exploring the optimized treatment path based on the policy intelligence.

### CROSS-REFERENCE TO RELATED APPLICATIONS

This application claims priority under 35 U.S.C. § 119 to Korean Patent Application Nos. 10-2021-0179326, filed on Dec. 15, 2021, and 10-2022-0086864, filed on Jul. 14, 2022, respectively, in the Korean Intellectual Property Office, the disclosures of which are incorporated by reference herein in their entireties.

### BACKGROUND

Embodiments of the present disclosure described herein relate to an artificial intelligence apparatus, and more particularly, relate to an artificial intelligence apparatus for planning and exploring an optimized treatment path, and an operation method thereof.

Medical artificial intelligence (AI) technology is developing in the order of diagnosing the presence of disease, predicting the patient condition, and exploring treatment methods. Currently, general medical AI is applied to diagnosis of disease, and recently, supervised or unsupervised deep learning technology is being used. In particular, the medical AI technology is widely used to detect abnormal areas in medical images such as CT, X-ray, Mill, etc. or to analyze continuous bio signals, and artificial intelligence technologies such as a CNN (convolutional neural network), a recurrent neural network, and an LSTM (long short-term memory) are mainly used.

In Korea, sepsis onset is predicted from an electronic medical record (EMR) using a graph convolution network (GCN), heart disease onset is predicted from cardiovascular bio signals using the LSTM and an artificial neural network (ANN), and arrhythmias and hypertension are diagnosed from blood vessel bio signals using an Autoencoder and the CNN. In overseas cases, by applying deep learning technologies to disease diagnosis, decision-making support, treatment, management, prevention, and surgery, health condition is quantified through the artificial intelligence that analyzes ultrasound images, and thus brain diseases such as stroke, dementia, and migraine are diagnosed. In addition, a model that can predict the HRD score, which is a measure of breast cancer diagnosis, is developed through the analysis of chest medical images, and comprehensive abnormalities of breast lesions are evaluated by analyzing an MM, mammography, and ultrasound. In addition, the artificial intelligence is being developed overseas to segment and visualize areas of interest in images such as liver lesions, lung nodules, and cardiac blood flow in medical images, and currently, the artificial intelligence is being developed to diagnose cancer and tumor lesions from radiation scan data of patients.

Among approved medical devices, the number of medical devices to which artificial intelligence technology related to disease diagnosis or lesion examination analysis is applied is increasing. In addition, just as medical institutions are developing technologies for predicting the possibility of developing heart failure in the future using electrocardiogram data, future health prediction technologies are being researched and developed. In particular, the medical AI technology may be applied to a treatment method exploration to find out which treatment method is most effective for a patient. The purpose of the treatment method exploration is to explore a series of treatment paths that ultimately improve a patient to the best condition, and for this, reinforcement learning technology may be used. The reinforcement learning is being used in various industries such as autonomous driving, detection of fraudulent insurance claims, and traffic control, but has not been studied much in the field of medical artificial intelligence.

### SUMMARY

Embodiments of the present disclosure provide an artificial intelligence apparatus for planning and exploring an optimized treatment path most suitable for a patient, and an operation method thereof.

According to an embodiment of the present disclosure, an artificial intelligence apparatus includes an episode conversion module that receives an electronic medical record (EMR) of a patient from an EMR database, converts the received EMR into an episode including a condition of the patient, a treatment method applied to the patient, and a treatment history of the patient, and stores the episode in an episode database, a patient condition predictive intelligence deep learning module that predicts a following condition of the patient after applying the treatment method to the patient, a local policy intelligence reinforcement learning module that performs reinforcement learning of a policy intelligence for planning an optimized treatment path for the patient based on the episode stored in the episode database by a development medical institution, an optimized treatment path exploration module that explores an optimized treatment path for the patient by using the policy intelligence, and a global policy intelligence management module that updates a global policy intelligence for planning the optimized treatment path based on the policy intelligence.

According to an embodiment of the present disclosure, a method of operating an artificial intelligence apparatus includes receiving an electronic medical record (EMR) of a patient and converting the received EMR into an episode including a condition of the patient, a treatment method applied to the patient, and a treatment history of the patient, performing deep learning of a patient condition predictive intelligence for predicting a following condition of the patient after applying the treatment method to the patient, performing reinforcement learning of a policy intelligence for planning an optimized treatment path for the patient based on the episode, exploring the optimized treatment path for the patient using the policy intelligence, updating a global policy intelligence for performing the reinforcement learning of the optimized treatment plan based on the policy intelligence, updating the global policy intelligence for policy intelligence reinforcement learning based on the policy intelligence, and outputting the optimized treatment path for the patient using the policy intelligence.

According to an embodiment of the present disclosure, a non-transitory computer-readable medium comprising a program code that, when executed by a processor, causes the processor to execute operations of receiving an electronic medical record (EMR) of a patient and converting the received EMR into an episode including a condition of the patient, a treatment method applied to the patient, and a treatment history of the patient, performing deep learning of a patient condition predictive intelligence for predicting a following condition of the patient after applying the treatment method to the patient, performing reinforcement learning of a policy intelligence for planning an optimized treatment path for the patient based on the episode, exploring the optimized treatment path for the patient using the policy intelligence, and updating a global policy intelligence for performing the reinforcement learning of the optimized treatment plan based on the policy intelligence.

### BRIEF DESCRIPTION OF THE FIGURES

The above and other objects and features of the present disclosure will become apparent by describing in detail embodiments thereof with reference to the accompanying drawings.

FIG. 1 is a diagram illustrating a mechanism of reinforcement learning for planning a treatment path.

FIG. 2 is a diagram illustrating an example of an optimized treatment path for a sepsis patient.

FIG. 3 is a diagram conceptually illustrating a process of deep learning a patient condition prediction intelligence corresponding to a patient model through an EMR and a process of reinforcement learning of a policy intelligence for planning an optimized treatment path.

FIG. 4 is a diagram conceptually illustrating a process of constructing an integrated EMR by integrating an EMR of a plurality of medical institutions, performing deep learning of a patient condition prediction intelligence corresponding to a patient model through an integrated EMR, and performing reinforcement learning of a policy intelligence for planning an optimized treatment path.

FIG. 5 is a diagram illustrating a method of performing reinforcement learning of a local policy intelligence for planning an optimized treatment path of each of a plurality of medical institutions.

FIG. 6 is a diagram illustrating an example of a configuration of an artificial intelligence apparatus for training a policy intelligence for an optimized treatment path plan and for exploring an optimized treatment path, according to an embodiment of the present disclosure.

FIG. 7 is a diagram illustrating a communication and remote exploration service of artificial intelligence apparatuses, according to an embodiment of the present disclosure.

FIG. 8 is a diagram illustrating an example of an operation method of an episode conversion module of FIG. 6.

FIG. 9 is a diagram illustrating an example of converting an EMR into an episode, according to a method of FIG. 8.

FIG. 10 is a diagram illustrating an example of an operation method of a patient condition predictive intelligence deep learning module of FIG. 6.

FIG. 11 illustrates an example of an operating method of a local policy intelligence reinforcement learning module of FIG. 6.

FIG. 12 is a diagram illustrating an example of an operation method of a global policy intelligence management module of FIG. 6.

FIG. 13 is a diagram illustrating an example of a method of planning and exploring an optimized treatment path for a patient using a global policy intelligence.

FIG. 14 is a diagram illustrating an example of a method of exploring an optimized treatment path for a patient in a remote medical institution.

### DETAILED DESCRIPTION

Hereinafter, embodiments of the present disclosure will be described in detail and clearly to such an extent that an ordinary one in the art easily implements the present disclosure.

Components that are described in the detailed description with reference to the terms “unit”, “module”, “block”, “˜er or ˜or”, etc. and function blocks illustrated in drawings will be implemented with software, hardware, or a combination thereof. For example, the software may be a machine code, firmware, an embedded code, and application software. For example, the hardware may include an electrical circuit, an electronic circuit, a processor, a computer, an integrated circuit, integrated circuit cores, a pressure sensor, an inertial sensor, a microelectromechanical system (MEMS), a passive element, or a combination thereof.

A treatment path of a disease may be defined as a series of processes that repeat the examination of the patient condition and medical action (treatment) to improve the patient condition until the patient is cured or dies. Deep learning technology may recommend a treatment method that can significantly improve a patient's following condition compared to a patient's current condition. However, even if the patient's following condition is greatly improved according to the recommended treatment method, when the subsequent treatment worsens the patient condition, the recommended treatment method is not the optimized treatment method. Therefore, the optimized treatment method is not a treatment that maximizes the level of immediate improvement, but a treatment method that makes the final condition of the patient the best.

To make the final condition of the patient the best, a continuous path of several treatments that can be performed step by step according to the change of the patient condition is required, and in the present disclosure, such a continuous path is defined as the optimized treatment path for the patient. For example, the treatment path may be expressed as a time series list of <patient's current condition, treatment method, patient's following condition, treatment history>.

An optimized treatment path plan may correspond to an exploration of sequential decision-making that maximizes a cumulative reward. In detail, the optimized treatment path plan is the training of successive treatment methods that will optimize the patient's final condition (i.e., maximize the cumulative reward) by repeatedly training the process of correcting the treatment method through trial and error that occurs whenever a treatment method is applied to a patient, and for this, a reinforcement learning method may be used.

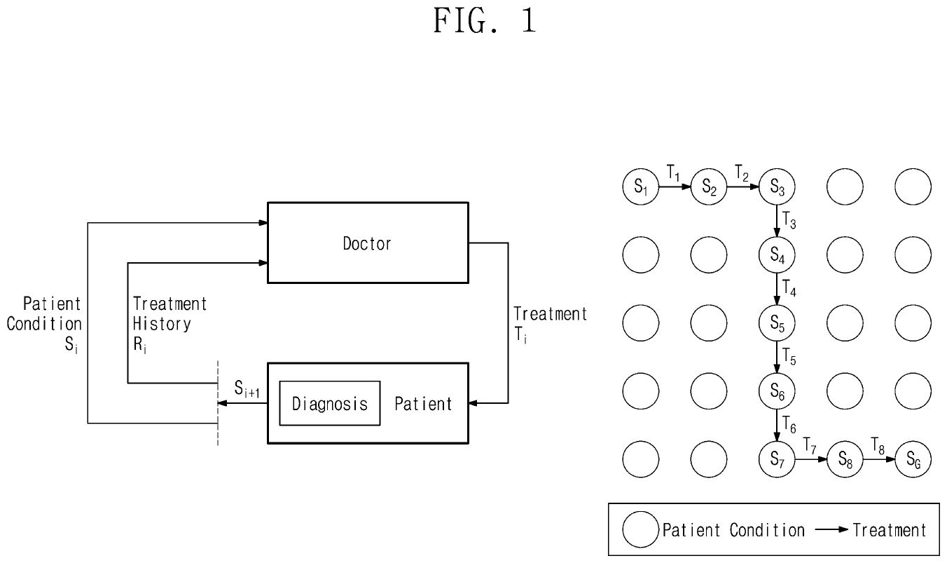

FIG. 1 illustrates a mechanism of reinforcement learning for planning an optimized treatment path. In this case, Sᵢ represents the patient condition at time i, Tᵢ represents the treatment method (unit treatment) to be applied to the patient at time i, and Rᵢ represents the treatment history (i.e., how is the patient condition at time i compared to time i−1) of the patient at time i. For example, the patient condition may include body temperature, heart rate, respiration rate, etc., the treatment method may include drug administration, and the treatment history may be expressed as any one of ‘survival’, ‘cure’, ‘death’, etc.

In FIG. 1, a doctor agent may select one treatment method Tᵢ according to the patient condition Sᵢ and may apply it to the patient, and the patient condition may be transitioned to the following condition Sᵢ₊₁. The patient's following condition Sᵢ₊₁ is a condition of the patient changed by treatment, and this changed condition may be quantified by examining the patient with a medical device. Thereafter, the doctor agent may determine a treatment history Rᵢ₊₁ by comparing the patient conditions Sᵢ and Sᵢ₊₁ at time i and time i+1. For example, treatment history may be quantified through means such as an e-SOFA score.

In this way, the doctor agent may train policy intelligence capable of planning the optimized treatment path by repeating the process of determining the treatment method Tᵢ at time i, based on the treatment history Rᵢ as the patient's condition Sᵢ at time i changes to the patient's condition Sᵢ₊₁ at time i+1 such that the cumulative treatment history is maximized (i.e., the cumulative reward is maximized). For example, the optimized treatment path may be a treatment path that allows the patient condition to reach a stable condition ‘G’. In this case, the final reward of the treatment path to reach the stable condition ‘G’ may be back-propagated to the previous steps.

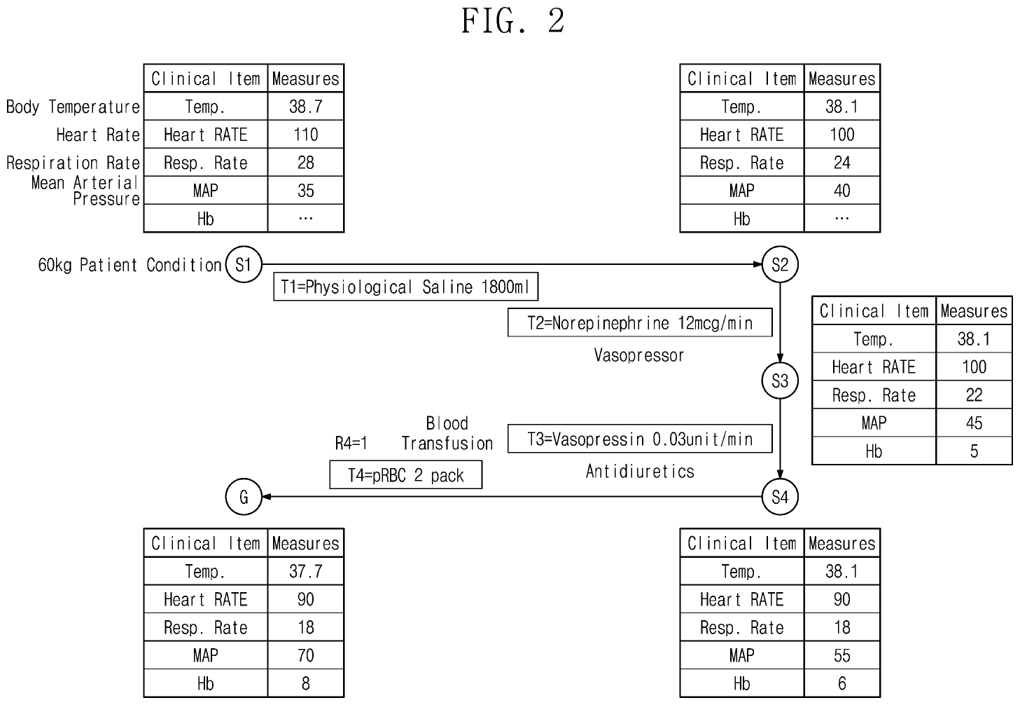

FIG. 2 illustrates an example of an optimized treatment path for a patient. S₁, S₂, S₃, and S₄ represent the patient condition for each time, and T₁, T₂, T₃, and T₄ represent the treatment method applied to the patient for each time. For example, the patient of FIG. 2 may be a sepsis patient, the patient condition for each time may be expressed through measures such as the patient's body temperature, heart rate, respiration rate, mean arterial pressure, etc., and the treatment method applied to the patient at each time may be administration of physiological saline, norepinephrine, vasopressin, or pRBC. In addition, the treatment history may be expressed as any one of whether the patient survived, whether the patient was cured, whether the patient died, or a difference in the e-SOFA score. The patient may reach the stable condition ‘G’ through the optimized treatment path consisting of T₁, T₂, T₃, and T₄.

In performing reinforcement learning for the optimized treatment path plan according to FIG. 1, since it is not possible to examine changes in patient condition after applying various treatment methods directly to actual patients, an artificial intelligence model that can replace actual patient is required. To this end, before performing reinforcement learning to plan the optimized treatment path according to an embodiment of the present disclosure, a patient model may be generated with patient condition prediction intelligence through deep learning based on the electronic medical record (EMR) of patients.

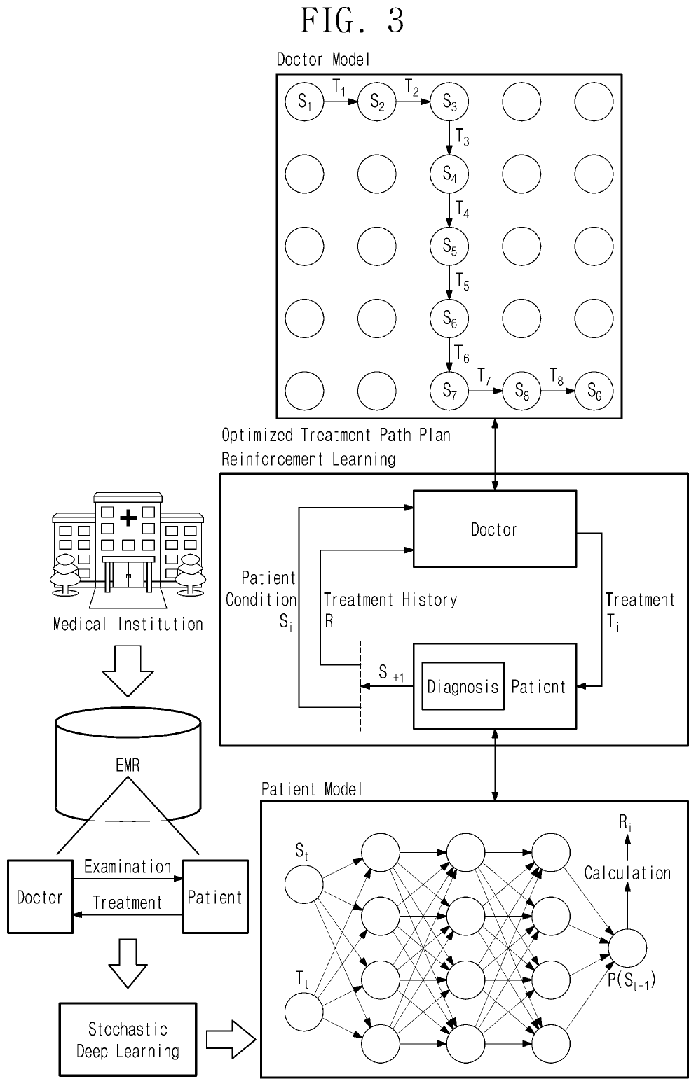

FIG. 3 conceptually illustrates a process of generating patient condition prediction intelligence through EMR-based deep learning and performing reinforcement learning of the policy intelligence for the optimized treatment path plan. Since policy intelligence to determine the most appropriate treatment for a patient is trained from this policy intelligence, the policy intelligence becomes a doctor model. For example, the patient model may be generated through stochastic deep learning based on the EMR. For example, when the treatment Tᵢ is performed on a patient in condition S₁ at time i, the probability that the patient condition transitions to Sᵢ₊₁ at time i+1 may be expressed as P(Sᵢ₊₁). In detail, when the same treatment Tᵢ is performed on several patients with the same patient condition as Sᵢ, only some patients may transition to Sᵢ₊₁, and the rest may transition to several other conditions, so the patient model may be modeled with probability distribution deep learning. In this case, the treatment history Rᵢ may be evaluated through Sᵢ₊₁. The patient model implemented in this way may serve as a patient in reinforcement learning for the optimized treatment path plan according to an embodiment of the present disclosure. The doctor model will perform reinforcement learning of policy intelligence by repeating attempts to find the optimized treatment method in the patient condition Sᵢ₊₁ in this patient model again. Accordingly, the doctor model may play a role as a doctor in reinforcement learning for optimized treatment path plan according to an embodiment of the present disclosure.

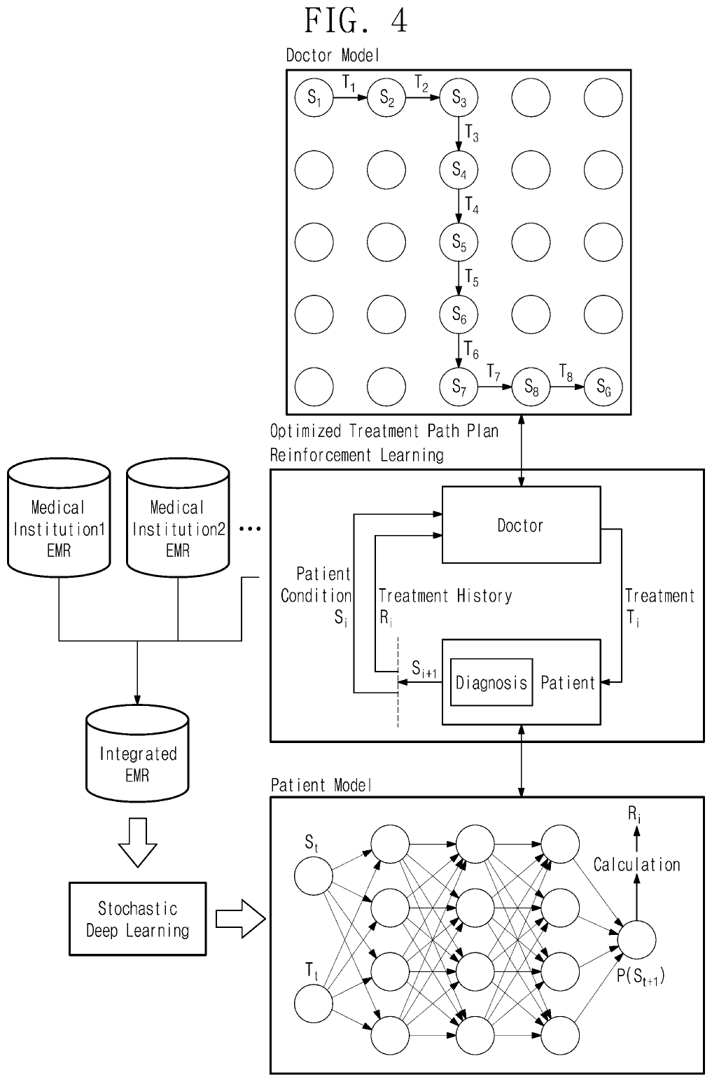

On the other hand, since the EMR may be biased toward a specific patient group according to the characteristics of a medical institution, a method capable of minimizing such bias is required. FIG. 4 conceptually illustrates a doctor model that constructs an integrated EMR by integrating the EMR of a plurality of medical institutions, generates a patient model through deep learning based on the integrated EMR, and performs reinforcement learning of the policy intelligence for the optimized treatment path plan. Referring to FIG. 4, a patient model having a probability distribution may be generated through stochastic deep learning based on the integrated EMR generated by integrating the EMR of each of a plurality of medical institutions (e.g., medical institutions 1 to 3).

However, in reality, since it may not be easy to integrate the EMR of each of the plurality of medical institutions, instead of using the integrated EMR, the present disclosure may use a method of integrating learning results by performing federation and synchronization for policy intelligence of each medical institution, after performing reinforcement learning of policy intelligence for the optimized treatment path plan individually by each medical institution. In this case, the policy intelligence for the optimized treatment path plan in which reinforcement learning is individually performed by each medical institution is called local policy intelligence.

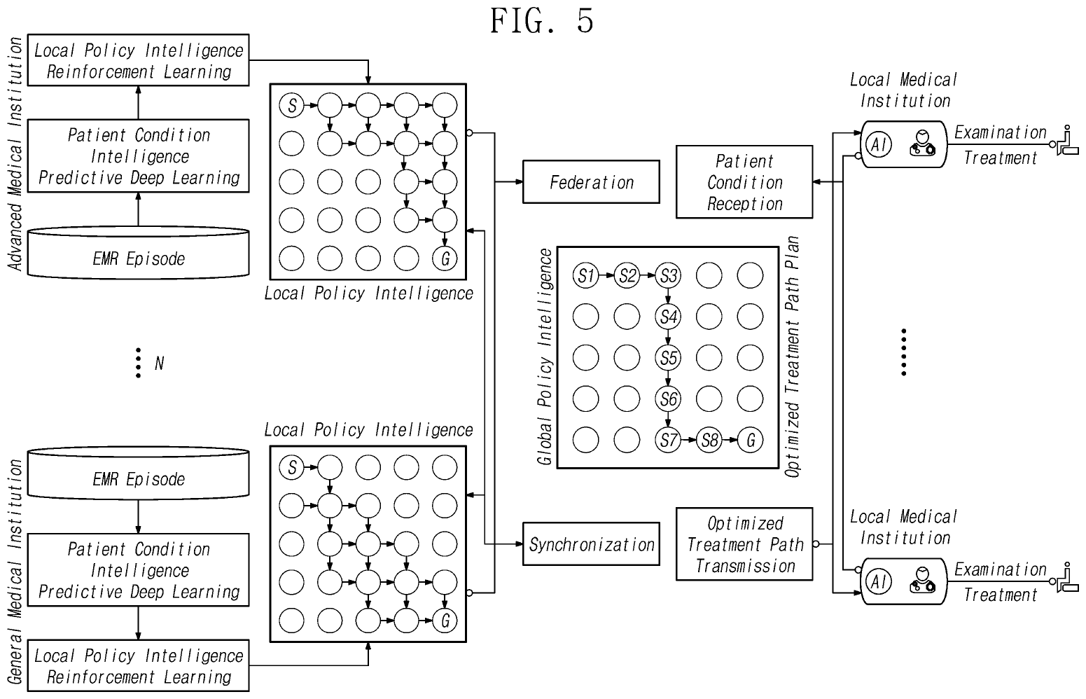

FIG. 5 illustrates a global policy intelligence reinforcement learning method based on local policy intelligence for the optimized treatment path plan for each of a plurality of medical institutions. FIG. 5 illustrates N medical institutions including advanced medical institutions and general medical institutions, and each medical institution may individually train policy intelligence (local policy intelligence) for the optimized treatment path plan based on the EMR. The federation and synchronization are repeatedly performed on local policy intelligences trained by each medical institution, such that global policy intelligence for the optimized treatment path plan that is not biased for a specific medical institution's patient group may be trained. For example, the global policy intelligence may be stored and managed in storage such as cloud storage.

Furthermore, referring to FIG. 5, the global policy intelligence trained in this way may be provided to various local medical institutions through a communication network such as an Internet. In this way, for patients in small medical institutions in various areas, as well as patients in advanced medical institutions and general medical institutions, it is possible to explore the optimized treatment path planned in the advanced medical institutions and the general medical institutions.

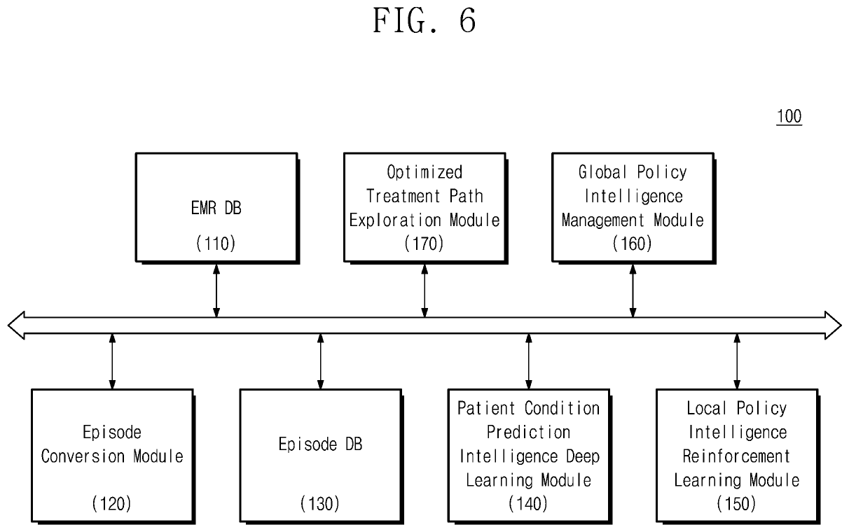

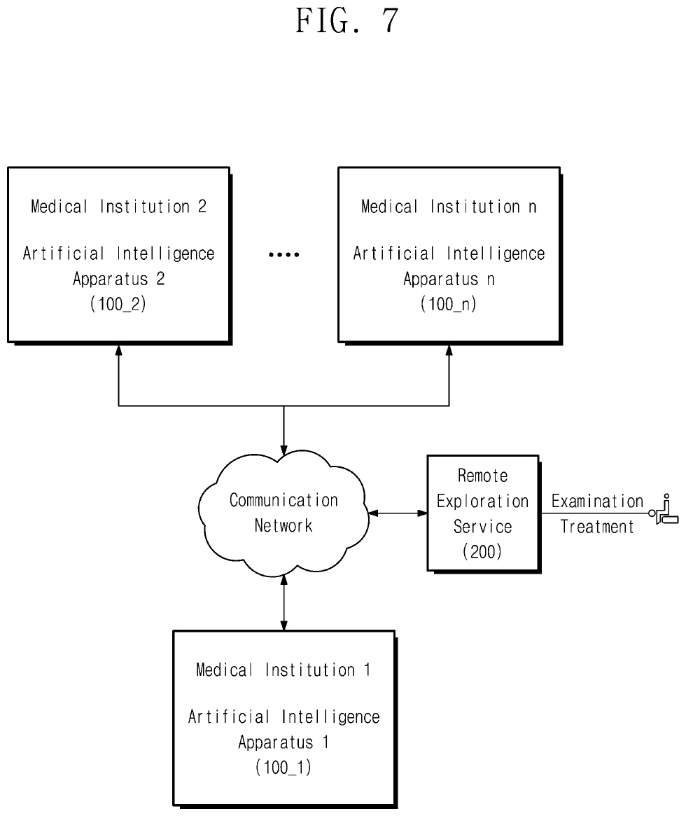

FIG. 6 illustrates an example of a configuration of an artificial intelligence apparatus 100 for optimized treatment path plan, according to an embodiment of the present disclosure. For example, a medical institution (e.g., a medical institution 1 and 2 of FIG. 7) may include one artificial intelligence apparatus 100. The artificial intelligence apparatus 100 includes an EMR database (DB) 110, an episode conversion module 120, an episode DB 130, a patient condition prediction intelligence deep learning module 140, a local policy intelligence reinforcement learning module 150, a global policy intelligence management module 160, and an optimized treatment path exploration module 170.

For example, functions of the artificial intelligence apparatus 100 may be implemented using hardware including combined logic, sequential logic, one or more timers, counters, registers, and/or state machines, complex instruction set computer (C SIC) processors such as one or more complex programmable logic device (CPLD), a field programmable gate array (FPGA), an application specific integrated circuit (ASIC), and x86 processors and/or a central processing unit (CPU) such as a reduced instruction set computer (RISC) such as ARM processors, a graphics processing unit (GPU), a neural processing unit (NPU), a tensor processing unit (TPU), an accelerated processing unit (APU), etc. or a combination thereof, to execute instructions stored in any type of memory (e.g., a NAND flash memory, a flash memory such as a low-latency NAND flash memory, a persistent memory (PMEM) such as a cross-grid nonvolatile memory, a memory with mass resistance change, a phase change memory (PCM), etc. or a combination thereof), software, or a combination thereof.

The EMR DB 110 is a database that stores an electronic medical record (EMR) of all patients. The EMR DB 110 stores the records associated with examination and treatment of all patients who have visited the medical institution in a time series format over time. The episode conversion module 120 may convert the EMR of each patient stored in the EMR DB 110 into a time series episode in the form of <patient's current condition, treatment method, patient's following condition, improvement degree>. The episode DB 130 is a database for storing the episode converted by the episode conversion module 120.

The patient condition prediction intelligence deep learning module 140 may perform deep learning with respect to an intelligent model capable of predicting the patient's following condition when a treatment method is applied to the patient (i.e., when unit treatment is performed). In the following description, the intelligent model capable of predicting the patient's following condition will be referred to as patient condition predictive intelligence. For example, the patient condition predictive intelligence may be a time series probability distribution model.

First, the patient's current condition is a result of past conditions and past treatment methods, and thus may be dependent on past conditions and treatment methods. Therefore, the patient condition prediction intelligence may be a time series deep learning model. In addition, the patient may be transitioned to various candidate conditions according to the treatment of the doctor, and a probability that the patient condition will transition to each of the candidate conditions may be different from each other. In addition, the probability that the patient condition will transition to each of the candidate conditions may be different for each patient. For example, even if the same treatment is performed on two patients with the same current condition, the following conditions of the two patients may be different from each other. Accordingly, the patient condition prediction intelligence may be a probability distribution model (e.g., a Bayesian probability distribution model) that trains the appearance distribution of each candidate condition for each patient.

The local policy intelligence reinforcement learning module 150 may perform reinforcement learning of policy intelligence (i.e., local policy intelligence) for planning the optimized treatment path based on episodes stored in the episode DB 130 of a medical institution. In addition, the local policy intelligence reinforcement learning module 150 federates the local policy intelligence of each medical institution through reinforcement learning, thereby contributing to updating the global policy intelligence for planning the optimized treatment path.

For example, the policy intelligence for planning the optimized treatment path may include the treatment method plan intelligence and the treatment history determination intelligence, and the global policy intelligence for planning the optimized treatment path may include the global treatment method plan intelligence and the global treatment history determination intelligence. The reinforcement learning performed by the local policy intelligence reinforcement learning module 150 is the same as described with reference to FIGS. 1 and 5.

The global policy intelligence management module 160 may update the global policy intelligence for planning and exploring the optimized treatment path based on the local policy intelligence in which reinforcement learning is performed through the local policy intelligence reinforcement learning module 150. To this end, the global policy intelligence management module 160 may receive a federation message and a synchronization message from an external medical institution, and may transmit the global policy intelligence to the external medical institution.

For example, the global policy intelligence management module 160 may transmit a learning result of a local policy intelligence received from an external medical institution in response to the federation message to a storage in which the global policy intelligence is located, and may update the global policy intelligence. In addition, the global policy intelligence management module 160 may transmit the updated global policy intelligence to the medical institution requesting synchronization in response to the synchronization message.

The optimized treatment path exploration module 170 may plan and explore the optimized treatment path for a patient using the policy intelligence or global policy intelligence trained through the local policy intelligence reinforcement learning module 150. Specifically, the optimized treatment path exploration module 170 may explore a treatment path for reaching a condition (e.g., a stable condition) in which the patient condition is the best. For example, the policy intelligence from which the optimized treatment path plan is trained may receive the patient's current condition as an input and may output the optimized treatment path. Also, the optimized treatment path exploration module 170 may provide the explored treatment path to various medical institutions.

In addition, according to embodiments, the above-described operations of the artificial intelligence apparatus 100 may be implemented with program codes stored in a non-transitory computer-readable medium. For example, the non-transitory computer-readable media may include magnetic media, optical media, or combinations thereof (e.g., a CD-ROM, a hard drive, a read-only memory, a flash drive, etc.).

FIG. 7 is a diagram illustrating a communication and remote exploration service of artificial intelligence apparatuses, according to an embodiment of the present disclosure. Referring to FIG. 7, medical institutions (medical institution 1, medical institution 2, . . . , and medical institution ‘n’) may have artificial intelligence apparatuses 100_1, 100_2, . . . , and 100_n, respectively. The configuration and operation of each of the artificial intelligence apparatuses 100_1, 100_2, . . . , and 100_n may be the same as those of the artificial intelligence apparatus 100 of FIG. 6.

Each medical institution (medical institution 1, medical institution 2, . . . , and medical institution ‘n’) may be connected through a communication network (e.g., the Internet), and the artificial intelligence apparatuses 100_1, 100_2, . . . , and 100_n may also communicate with each other through the communication network. For example, the global policy intelligence may be stored in the cloud storage on the Internet. As described with reference to FIG. 6, each of the artificial intelligence apparatuses 100_1, 100_2, . . . , and 100_n may perform reinforcement learning of the local policy intelligence for the optimized treatment path plan, and each local policy intelligence may be used to perform reinforcement learning of the global policy intelligence and to update the global policy intelligence.

Through the communication network directly with medical institutions (medical institution 1, medical institution 2, . . . , and medical institution ‘n’), a remote medical institution may transmit the patient's EMR to each medical institution through a remote exploration service 200. After that, the artificial intelligence apparatus of each medical institution may explore the optimized treatment path based on the received policy intelligence for the patient, and may transmit the explored result back to the remote medical institution through the remote exploration service 200. Accordingly, the doctor of the remote medical institution may treat the patient according to the optimized treatment path.

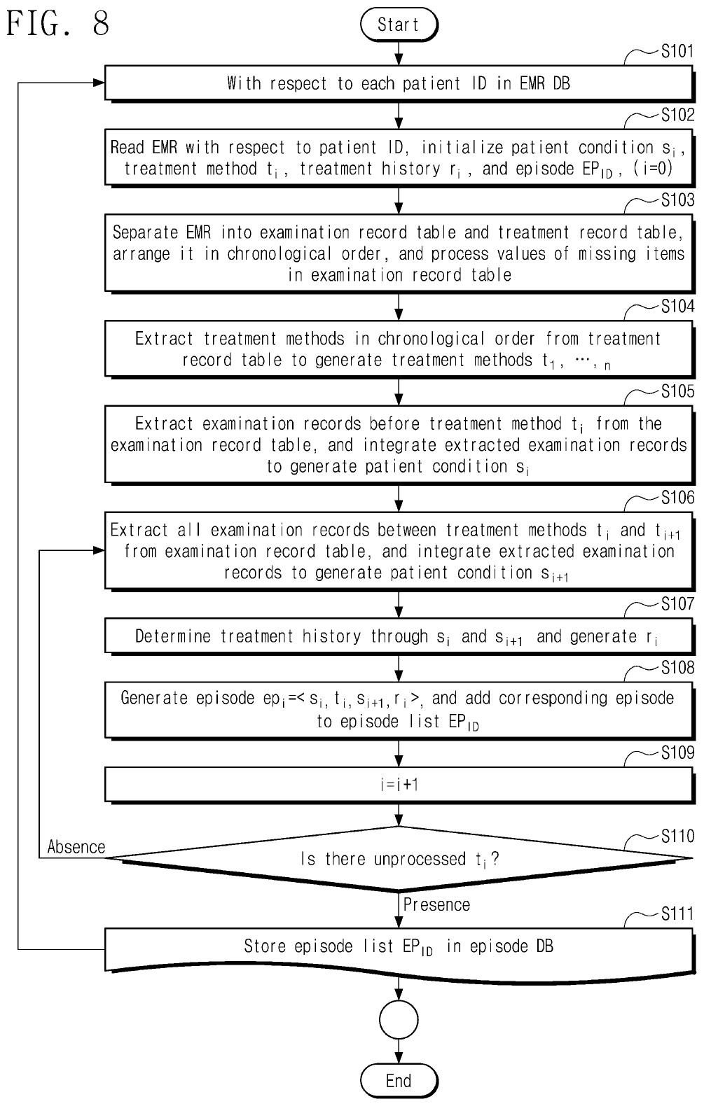

FIG. 8 illustrates an example of an operation method of the episode conversion module 120 of FIG. 6. Hereinafter, it will be described with reference to FIG. 6 together with FIG. 8.

In operation S102, the episode conversion module 120 may read the EMR with respect to each patient ID in the EMR DB in operation S101, and may initialize the patient condition, treatment method, treatment history, and episode list. For example, the initialized patient condition, treatment method, treatment history, and episode may be represented by identifiers s₁, t₁, r₁, and ep₁, respectively. In operation S103, the episode conversion module 120 may separate the EMR into an examination record table and a treatment record table, may arrange records in the two tables in chronological order, and may process values of missing items in the examination record table. For example, the missing values in the examination record table may be replaced with an average value, a mode value, an interpolation value, etc. of the corresponding EMR items.

In operation S104, the episode conversion module 120 may extract and integrate treatment records in a similar time period from the treatment record table, and may assign identifiers such as t₁, . . . with respect to all treatment methods. In operation S105, the episode conversion module 120 may extract the examination records before the treatment method tᵢ (i.e., earlier than the time corresponding to the treatment method tᵢ) from the examination record table, and may integrate the extracted examination records into one to generate a patient condition identifier sᵢ. In operation S106, the episode conversion module 120 may extract the examination records between the treatment methods tᵢ and tᵢ₊₁ from the examination record table, and may integrate the extracted examination records into one to generate a patient condition identifier sᵢ₊₁. For example, when blood pressure measurement values are recorded twice (“150 mmHG” and “140 mmHG”) in the examination record integration, the values may be integrated as the latest value/first value/average value, etc. In operation S107, the episode conversion module 120 may determine the treatment history from the examination record table and may generate an identifier n. In this case, the treatment history may be ‘survival’, ‘cure’, ‘death’, e-SOFA score, etc.

In operation S108, the episode conversion module 120 may generate an identifier epᵢ with respect to one episode <sᵢ, tᵢ, sᵢ₊₁, rᵢ>, and may add the corresponding episode to an episode list ep_{ID}. For example, the episode ep_{ID} may be expressed as {<s₁, t₁, s₂, r₁>, <s₂, t₂, s₃, r₂> . . . }. In operation S109, the episode conversion module 120 sets i to i+1 to generate the following episode. In operation S110, it may be repeatedly processed for all treatment methods.

In operation S111, the episode conversion module 120 may store a list of all episodes in the episode DB 130. The episode conversion module 120 may perform the above-described operations S101 to S111 based on the EMRs of all patients, and the EMRs of all patients may be converted into a corresponding episode list.

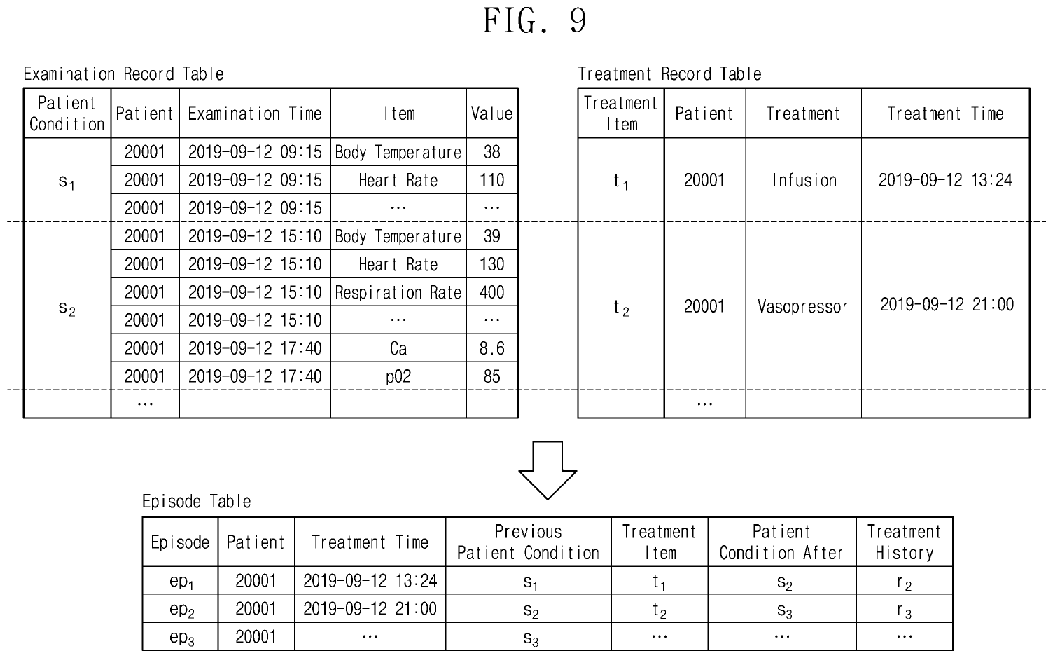

FIG. 9 is a diagram illustrating an example of converting an EMR into an episode, according to a method of FIG. 8. The EMR may include examination record data and treatment record data. The examination record data is a record representing the condition of the patient who has been tested, and the treatment record data is a record representing the medical treatment applied to the patient, such as medication and treatment. Hereinafter, it will be described with reference to FIGS. 6 and 8 together with FIG. 9.

As described with reference to FIG. 8, the episode conversion module 120 may separate the EMR into the examination record table including examination record data and the treatment record table including treatment record data, and may arrange each table in chronological order. In this case, the patient condition may be extracted from the examination record table based on the treatment time of the treatment record table.

For example, in the case of patient 20001, based on the treatment time (2019-09-12 13:24) corresponding to treatment method t₁ (infusion administration), the body temperature 38 and heart rate 110 measured at the previous time (2019-09-12 09:15) in the examination record table become the condition s₁. As in the above description, based on the treatment time (2019-09-12 21:00) corresponding to treatment method t₂ (administration of vasopressor), the body temperature 39 and heart rate 130 measured at the previous time (2019-09-12 15:10, 17:40) in the examination record table become condition s₂. The treatment history may be recorded as one of ‘survival’, ‘cure’, and ‘death’, and both r₁ and r₂ may be recorded as ‘survival’.

The episode may be generated based on the thus generated condition, treatment method, and treatment history, and the episode may be expressed as a time series list of <patient's current condition, treatment method, patient's following condition, treatment history>. For example, the episode ep₁ associated with the patient 20001 may be expressed as <s₁, t₁, s₂, r₁>, and episode ep₂ may be expressed as <s₂, t₂, s₃, r₂>.

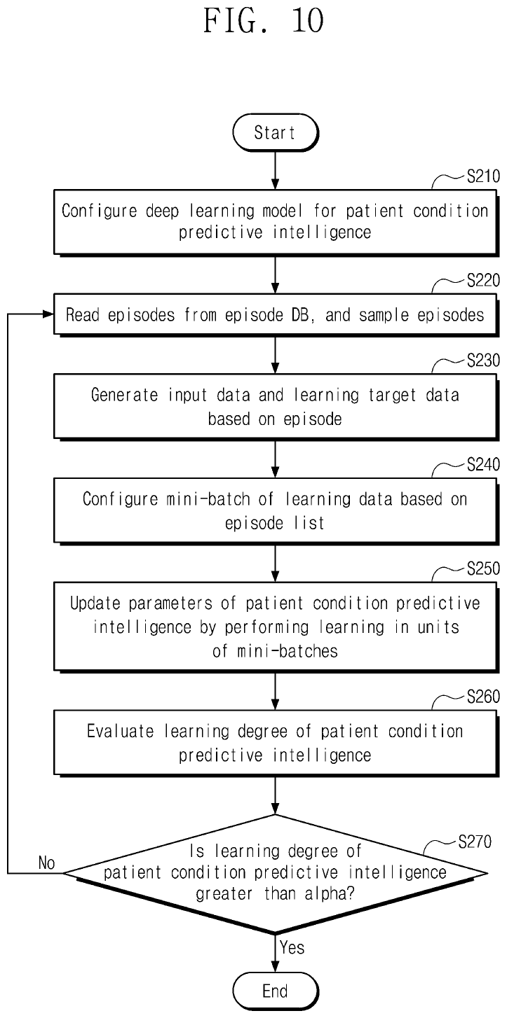

FIG. 10 illustrates an example of an operation method of the patient condition predictive intelligence deep learning module 140 of FIG. 6. As described with reference to FIG. 6, the patient condition predictive intelligence deep learning module 140 may train the patient condition predictive intelligence. Hereinafter, it will be described with reference to FIG. 6 together with FIG. 10.

In operation S210, the patient condition predictive intelligence deep learning module 140 may configure a deep learning model for the patient condition predictive intelligence. The patient condition prediction intelligence may be configured to make predictions with uncertainty even for non-deep learning patient conditions, and configured to be predicted with different probability distributions with respect to similar patient condition time series. For example, the RNN and the LSTM may be used as the time series model, and a multiple Bayesian model may be used as the probability distribution model, but the present disclosure is not limited thereto.

In operation S220, the patient condition predictive intelligence deep learning module 140 may read the episodes from the episode DB 130, and may sample the episodes. In operation S230, the patient condition predictive intelligence deep learning module 140 may generate input data and learning target data based on the episode. For example, as illustrated in FIG. 9, when it is assumed that the patient's episodes ep₁, ep₂, . . . epₙ is expressed as <s₁, t₁, s₂, r₁>, <s₂, t₂, s₃, r₂>, . . . , and <sₙ, tₙ, sₙ₊₁, rₙ>, the input data is <s₁, t₁, s₂, r₁>, <s₂, t₂, s₃, r₂>, . . . , and <sₙ, tₙ, -, ->, and the learning target data is sₙ, and through this, e-SOFA score and rₙ may be calculated.

In operation S240, the patient condition predictive intelligence deep learning module 140 may configure a mini-batch of learning data to perform efficient deep learning. In operation S250, the patient condition predictive intelligence deep learning module 140 may update the time series probability distribution deep learning parameters of the patient condition predictive intelligence by performing learning in units of mini-batches. In operation S260, the patient condition predictive intelligence deep learning module 140 may evaluate the learning degree of the patient condition predictive intelligence, and in operation S270, the patient condition predictive intelligence deep learning module 140 may compare the learning degree with a preset threshold ‘alpha’. When the learning degree is less than or equal to the preset threshold ‘alpha’, the patient condition predictive intelligence deep learning module 140 may perform operations S220 to S260 again, and when the learning degree is greater than the preset threshold ‘alpha’, the patient condition predictive intelligence deep learning module 140 in operation S280 may store the patient condition predictive intelligence as the patient model of the local policy intelligence reinforcement learning module 150 of FIG. 6.

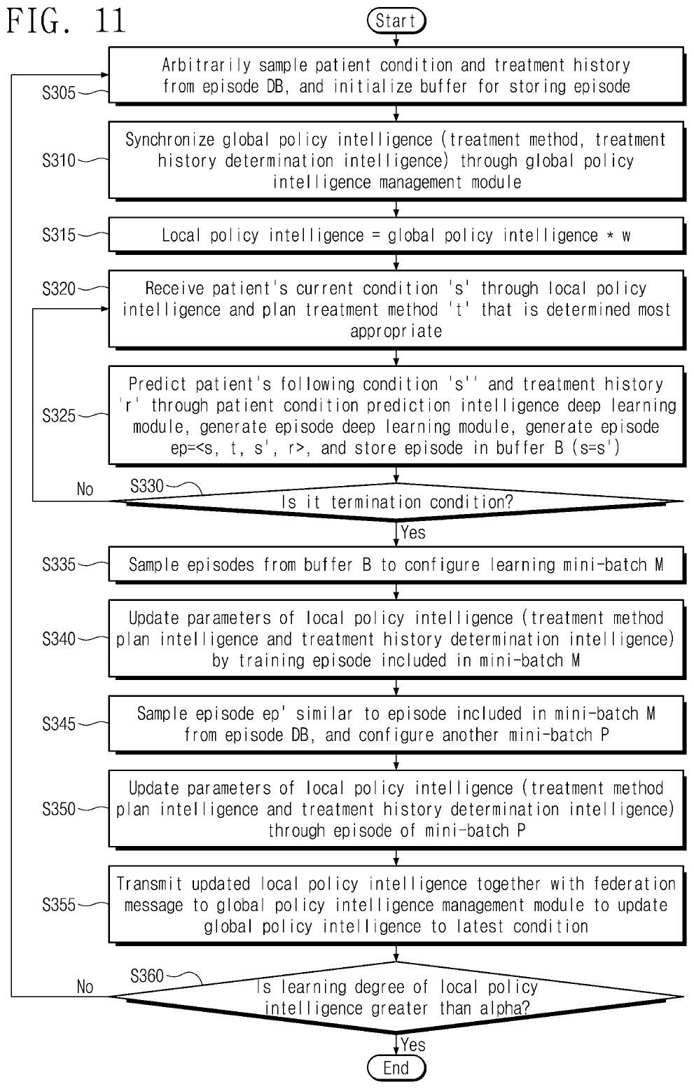

FIG. 11 illustrates an example of an operating method of the local policy intelligence reinforcement learning module 150 of FIG. 6. Hereinafter, it will be described with reference to FIG. 6 together with FIG. 11.

In operation S305, the local policy intelligence reinforcement learning module 150 may arbitrarily sample a patient condition ‘s’ and a treatment history ‘r’ from the episode DB 130, and may initialize a buffer for storing the episode to be subjected to reinforcement learning. In operation S310, the local policy intelligence reinforcement learning module 150 may synchronize the local policy intelligence (i.e., the treatment method plan intelligence and the treatment history determination intelligence) with the global policy intelligence (i.e., the global treatment method plan intelligence and the global treatment history determination intelligence) through the global policy intelligence management module 160. Accordingly, the local policy intelligence reinforcement learning module 150 may perform reinforcement learning based on the latest treatment method plan intelligence and the latest treatment history determination intelligence.

In operation S315, the local policy intelligence reinforcement learning module 150 may adjust a reflection ratio to the global policy intelligence by a specified weight ‘w’. In this case, the weight ‘w’ may represent a ratio of reflecting the global policy intelligence to the local policy intelligence, and may be represented as 0≤w≤1. When the weight value is ‘1’, the parameters constituting the global policy intelligence are reflected as they are, but when the weight value is ‘0’, the parameters constituting the global policy intelligence are not reflected at all, so the parameters of the local policy intelligence are used as they are.

In operation 320, the local policy intelligence reinforcement learning module 150 may receive the patient's current condition ‘s’ and may plan several treatment methods T through the treatment method plan intelligence, may determine how appropriate this treatment is through the treatment history determination intelligence. In operation S325, the local policy intelligence reinforcement learning module 150 may input the patient's current condition ‘s’ and the planned treatment method ‘t’ to the patient condition prediction intelligence deep learning module 140 of FIG. 6, may generate the episode ep=<s, t, s′, r> and may store it in a buffer, based on s, t, s′, r that predict the patient's following condition ‘s′’ and the treatment history ‘r’. It is assumed that the ‘s’ is the same as ‘s′’ (s=s′) to generate an episode for the patient's following condition ‘s′’. Accordingly, episodes generated in the buffer may be continuously accumulated. In operation 330, it is determined whether or not the process of generating a list of treatment episodes for the patient condition in operation 325 is ended. The end condition of the process of generating the episode is when the treatment history is ‘death’, ‘cure’, or more than a specified number of repetitions. In operation 330, when the end condition is satisfied, the process of generating a list of treatment episodes for the patient condition is ended.

In operation S335, the local policy intelligence reinforcement learning module 150 may sample the episodes from the buffer to configure a learning mini-batch ‘M’. That is, the mini-batch ‘M’ may include a plurality of episodes. In operation S340, the local policy intelligence reinforcement learning module 150 may update the parameters of the local policy intelligence (the treatment method plan intelligence and the treatment history determination intelligence) by training the episodes included in the mini-batch ‘M’. Since the mini-batch ‘M’ is episodes with respect to the treatment path planned by the local policy intelligence synchronized by the global policy intelligence, this updating of parameters of the local policy intelligence will train a new local policy intelligence.

Specifically, the episodes included in the mini-batch ‘M’ may be based on the results planned in operation S320. Therefore, when it is determined that the weights (i.e., the reflection ratio of parameters of global policy intelligence) associated with the treatment method plan intelligence and the treatment history determination intelligence set in operation S315 are appropriate, the local policy intelligence reinforcement learning module 150 may strengthen the parameters of the treatment method plan intelligence and the treatment history determination intelligence, and may weaken the parameters when it is determined that the weights are inappropriate.

For example, the treatment method plan intelligence may be updated in the direction targeted by the treatment history determination intelligence, and the treatment history determination intelligence may be updated to plan a treatment path that leads to a patient's full recovery. For example, in operation S340, the parameter update for the treatment method plan intelligence and the treatment history determination intelligence may be performed according to various reinforcement learning methods such as an actor and critic, but the present disclosure is not limited thereto.

On the other hand, the update result of the treatment history determination intelligence in operation S340 may be dependent on the patient condition prediction intelligence in operation S325. Therefore, the local policy intelligence reinforcement learning module 150 should additionally determine whether the updated treatment history determination intelligence matches the treatment performed in the doctor model learned so far.

To this end, in operation S345, the local policy intelligence reinforcement learning module 150 may sample an episode ep′ similar to the episode ep included in the mini-batch ‘M’ from the episode DB 130, and may configure another mini-batch ‘P’. The episode ep′ may indicate the actual treatment method and the treatment result performed by a doctor at a medical institution with respect to a patient condition similar to that in the corresponding episode ‘ep’.

Then, in operation S350, the local policy intelligence reinforcement learning module 150 may further update the parameters of the treatment history determination intelligence through the episode of the mini-batch ‘P’. Therefore, the updated treatment history determination intelligence will allow the treatment method plan intelligence to plan future treatment methods similar to that of a doctor. Since the mini-batch ‘P’ is based on the actual medical record of the doctor, the parameters of the treatment history determination intelligence may be updated in a direction that matches the treatment performed actually by the doctor.

For example, parameters of treatment history determination intelligence may be updated through Equation 1 below. In Equation 1, the treatment history determination intelligence may be expressed as a reinforcement learning model Q.

$${Q}^{\text{?} + 1} = {argmin}_{Q} ⌊ ⌊ α \cdot ( {𝔼}_{s \sim M , t \sim μ ( t \vert s )} ⌊ Q ( s , t ) ⌋ - {𝔼}_{{s}^{\text{?}} \sim P , t - \text{?} ( t \vert {s}^{\text{?}} )} ⌊ Q ( {s}^{\text{?}} , t ) ⌋ ) + 0.5 \cdot {TD}_{{Q}^{\text{?}}} \text{?} ( n ) ⌋ ⌋ \qquad [ Equation 1 ]$$

$$\text{?} \text{indicates text missing or illegible when filed}$$

Here, ‘α’ is a specified weight that can be adjusted, ‘M’ is a learning mini-batch including a plurality of episodes, and ‘P’ is a mini-batch including episodes similar to those included in the mini-batch ‘M’ among records treated by a doctor at an actual medical institution. S is the patient condition predicted through the patient condition prediction intelligence, and \tilde{S} is a patient condition similar to S, which is the actual patient condition observed at a medical institution.

[symbol] _{s˜M,t˜μ(t|s)}[Q(s,t)] is the expected value of the reward calculated through reinforcement learning Q when the treatment t is attempted for the patient condition S, and [symbol] _{s˜P,t˜π(t|s)}[Q(\tilde{S},t)] is the expected value of the reward calculated through reinforcement learning Q when treatment t is attempted for an actual patient condition \tilde{S}. For example, the reward may correspond to the degree to which a patient condition is improved.

In detail, [symbol] _{s˜M,t˜μ(t|s)}[Q(s,t)]− [symbol] _{s˜P,t˜π(t|s)}[Q(\tilde{s},t)] represents the difference between the expected value of the reward corresponding to the patient condition S and the treatment t through reinforcement learning Q, and the expected reward value corresponding to the similar patient condition \tilde{S} and treatment \tilde{t}. When reinforcement learning Q plans treatment t for patient condition S, and when treatment t is planned for patient condition \tilde{S} in an actual medical institution, the difference between the two expected values may be minimized, so the parameters may be updated such that the corresponding reinforcement learning Q is reinforced in the direction of selection.

In contrast, when the reinforcement learning Q plans the treatment t in the condition S but the treatment t is not performed on the patient condition \tilde{S} in an actual medical institution, the difference between the two expected values may increase, and the parameters of the corresponding reinforcement learning Q may be updated such that they are reinforced in a non-selected direction.

TD_{Q}_{^{k}}(n) represents n temporal difference learning among reinforcement learning algorithms. Therefore, according to Equation 1, the reinforcement learning Q that can minimize the difference between the expected value of reward when treatment is planned for the patient condition through reinforcement learning and the expected value of reward when the medical institution plans treatment for the actual patient condition may be selected (i.e., selecting the Q that minimizes both differences, argmin_{Q}).

In operation S355, the local policy intelligence reinforcement learning module 150 may transmit the updated local policy intelligence (the treatment method plan intelligence and the treatment history determination intelligence) together with the federation message to the global policy intelligence management module 160. The transmitted local policy intelligence may be used to update the global policy intelligence to the latest condition.

In operation S360, the local policy intelligence reinforcement learning module 150 may compare the learning degree for the treatment method plan intelligence and the treatment history determination intelligence with the preset threshold ‘alpha’. When the degree of reinforcement learning is less than or equal to the preset threshold ‘alpha’, the local policy intelligence reinforcement learning module 150 may return to operation S305 to perform reinforcement learning on the treatment method plan intelligence and the treatment history determination intelligence again, and when the learning degree is higher than the preset threshold ‘alpha’, the local policy intelligence reinforcement learning module 150 may end the learning.

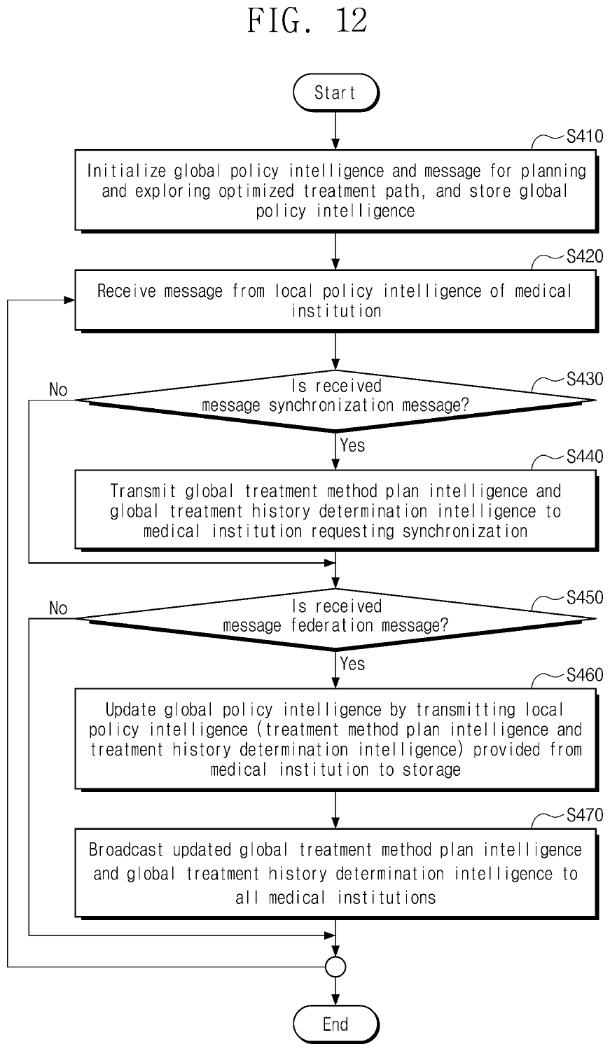

FIG. 12 illustrates an example of an operation method of the global policy intelligence management module 160 of FIG. 6. As described with reference to FIG. 6, the global policy intelligence for planning and exploring the optimized treatment path may be divided into the global treatment method plan intelligence and the global treatment history determination intelligence and may be managed. Hereinafter, it will be described with reference to FIG. 6 together with FIG. 12.

In operation S410, the global policy intelligence management module 160 may initialize the global policy intelligence and message for planning and exploring the optimized treatment path, and may store the initialized global policy intelligence in storage (e.g., the cloud of the communication network). Operation S410 may be performed only when training the global policy intelligence for the first time, and is not performed when the global policy intelligence has already been trained.

In operation S420, the global policy intelligence management module 160 may receive a message from the local policy intelligence of another medical institution. First, in operation S430, the global policy intelligence management module 160 may determine whether the received message is a synchronization message. When the received message is the synchronization message, in operation S440, the global policy intelligence management module 160 may transmit the global treatment method plan intelligence and the global treatment history determination intelligence to the medical institution requesting the synchronization. When the received message is not the synchronization message, in operation S450, the global policy intelligence management module 160 may determine whether the received message is a federation message.

When the received message is the federation message, in operation S460, the global policy intelligence management module 160 may update the global policy intelligence by transmitting the treatment method plan intelligence and the treatment history determination intelligence provided from the medical institution to the storage. Then, in operation S470, the global policy intelligence management module 160 may broadcast the updated global treatment method plan intelligence and global treatment history determination intelligence to all medical institutions in response to the federation message. This notifies all medical institutions that the global policy intelligence for optimized treatment path plan is updated. The medical institution receiving this message may perform the local policy intelligence reinforcement learning module 150. When the received message is not the federation message, the operation of the global policy intelligence management module 160 may be terminated.

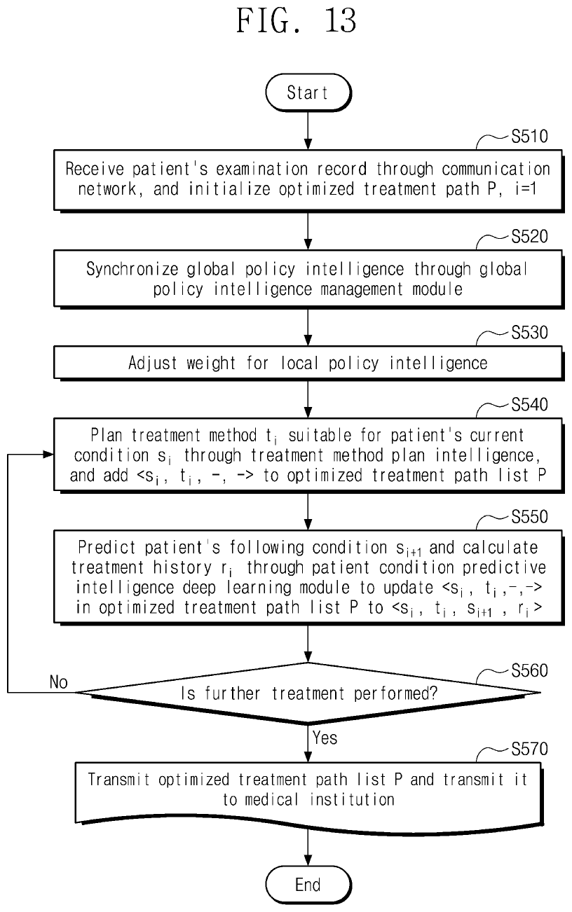

FIG. 13 is a diagram illustrating an example of a method for planning and exploring an optimized treatment path for a patient using a global policy intelligence. Hereinafter, it will be described with reference to FIG. 6 together with FIG. 13.

In operation S510, a medical institution may receive the patient's examination record through a communication network, and may initialize the optimized treatment path ‘P’ through the artificial intelligence apparatus 100. In operation S520, the medical institution may synchronize the global policy intelligence (the treatment method plan intelligence and the treatment history determination intelligence) through the global policy intelligence management module 160. In other words, the medical institution may utilize the latest global policy intelligence to plan the optimized treatment path.

In operation S530, the medical institution may adjust the weight ‘w’ for local policy intelligence (the treatment method plan intelligence and the treatment history determination intelligence) through the local policy intelligence reinforcement learning module 150. In this case, the weight ‘w’ may represent the ratio of reflecting the global treatment method plan intelligence and the global treatment history determination intelligence to the local policy intelligence, respectively, and may have a value of 0≤w≤1. When the value of the weight is ‘1’, the parameters constituting the global policy intelligence are reflected as they are, and when the value of the weight is ‘0’, the parameters constituting the global policy intelligence are not reflected at all.

In operation S540, the medical institution may plan the treatment method tᵢ suitable for the patient's current condition ‘s’ through the reinforcement learning treatment method plan intelligence, and may add sᵢ and tᵢ to the optimized treatment path list ‘P’. For example, the ‘P’ may appear as <sᵢ, tᵢ, -, ->. In this case, the treatment history determination intelligence may determine the treatment history such that the treatment method plan intelligence may plan the treatment method that will give the best treatment history when planning the treatment method.

In operation S550, the medical institution may predict the patient's following condition sᵢ₊₁ and may calculate the treatment history rᵢ using the patient condition predictive intelligence trained through the patient condition predictive intelligence deep learning module 140. For example, the input data of the patient condition prediction intelligence is <s₁, t₁, s₂, r₁>, <s₂, t₂, s₃, r₂>, <sₙ₋₁, tₙ₋₁, sₙ, rₙ> and tₙ, and the learning target data are sₙ₊₁ and rₙ. This means that the patient condition predictive intelligence trains the case of the patient who transitions to sₙ₊₁ and rₙ when treatment tₙ is performed on the patient whose past treatment path episodes are <s₁, t₁, s₂, r₁>, <s₂, t₂, S₃, r₂>, . . . , and <sₙ₋₁, tₙ₋₁, sₙ, rₙ>. In operation S540, one <sᵢ, tᵢ, -, -> in the optimized treatment path list ‘P’ may be updated to <sᵢ, tᵢ, sᵢ₊₁, rᵢ>.

In operation S560, the medical institution may determine whether further treatment is performed. For example, when the treatment history rᵢ is ‘death’ or ‘cure’, no further treatment is performed, and in operation S570, the artificial intelligence apparatus 100 may output the optimized treatment path ‘P’ or transmit it to a medical institution. In contrast, when the treatment history rᵢ is ‘survival’, further treatment is performed, and the medical institution may perform again from operation S540, where i becomes i+1.

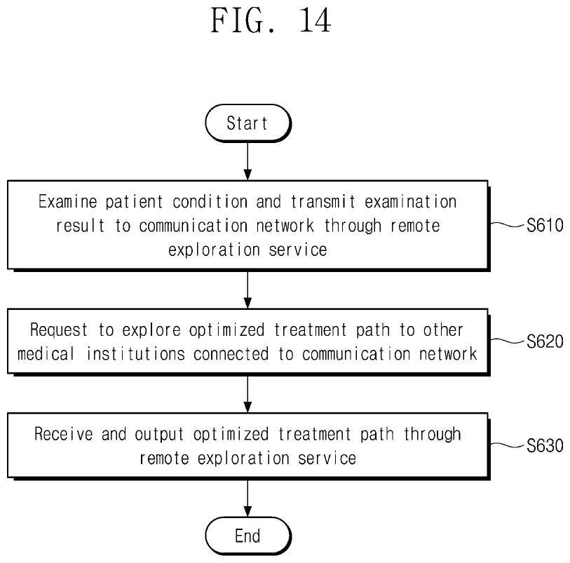

FIG. 14 is a diagram illustrating an example of a method of exploring an optimized treatment path for a patient in a remote medical institution. In operation S610, the remote medical institution may examine the patient condition and may transmit the examination result to a communication network (e.g., the Internet) through a remote exploration service (200 of FIG. 7). In operation S620, the remote medical institution may request to explore an optimized treatment path to other medical institutions connected to the communication network. In operation S630, the remote medical institution may receive and output the optimized treatment path planned from the global policy intelligence or the local policy intelligence of another medical institution through the remote exploration service.

According to an embodiment of the present disclosure, an optimized treatment path for a patient may be automatically explored through medical artificial intelligence. In addition, multiple medical institutions with electronic medical records (EMR) may perform reinforcement learning of global policy intelligence for optimized treatment path plan in collaboration with each other. Furthermore, according to an embodiment of the present disclosure, various medical institutions may remotely share and utilize global policy intelligence for plan and exploration of the optimized treatment path.

According to an embodiment of the present disclosure, there are the following advantages. First, the optimized treatment path planning intelligence may be trained based on the treatment records of various medical institutions, and the most suitable treatment plan path for the patient may be explored based on this plan intelligence. Second, this planning and exploration may alleviate the disparities in medical services between regions due to shortages of medical personnel and infrastructure. In addition, clinic-level medical institutions may remotely share and utilize high-quality global policy intelligence, thereby minimizing patient bias in specific medical institutions. Third, the intelligence for planning and exploring the optimized treatment path according to the embodiment of the present disclosure may be used as a medical service model that may coexist with each other through role sharing among general medical institutions, small and medium-sized medical institutions, and clinic-level medical institutions, thereby strengthening the public nature of medical care. Fourth, the average treatment time per a patient may be shortened and the average number of patients treated per a doctor may be increased, thereby maximizing the utilization and efficiency of medical resources. Fifth, artificial intelligence medical services using domestic medical big data may be internationalized.

According to an embodiment of the present disclosure, multiple medical institutions with an electronic medical records (EMR) may perform reinforcement learning of policy intelligence for planning and exploring the optimized treatment path in collaboration with each other.

Furthermore, according to an embodiment of the present disclosure, various medical institutions may remotely share and utilize global policy intelligence for planning and exploring the optimized treatment path.

The above description refers to embodiments for implementing the present disclosure. Embodiments in which a design is changed simply or which are easily changed may be included in the present disclosure as well as an embodiment described above. In addition, technologies that are easily changed and implemented by using the above embodiments may be included in the present disclosure. While the present disclosure has been described with reference to embodiments thereof, it will be apparent to those of ordinary skill in the art that various changes and modifications may be made thereto without departing from the spirit and scope of the present disclosure as set forth in the following claims.

What is claimed is:

1. An artificial intelligence apparatus comprising:
an episode conversion module configured to receive an electronic medical record (EMR) of a patient from an EMR database, to convert the received EMR into an episode including a condition of the patient, a treatment method applied to the patient, and a treatment history of the patient, and to store the episode in an episode database;
a patient condition predictive intelligence deep learning module configured to train a patient condition predictive intelligence for predicting a following condition of the patient after applying the treatment method to the patient;
a local policy intelligence reinforcement learning module configured to perform reinforcement learning of a policy intelligence for exploring an optimized treatment path for the patient based on the episode stored in the episode database;
an optimized treatment path exploration module configured to output the optimized treatment path for the patient by using the policy intelligence; and
a global policy intelligence management module configured to update a global policy intelligence for exploring the optimized treatment path based on the policy intelligence.

2. The artificial intelligence apparatus of claim 1, wherein the episode is a time series having an order of a first condition of the patient, the treatment method applied to the patient, a second condition of the patient after applying the treatment method, and the treatment history of the patient.

3. The artificial intelligence apparatus of claim 1, wherein the patient condition predictive intelligence is a time series mixed probability distribution model that predicts a plurality of conditions that can be resulted in when the treatment method is applied to the patient and a probability that each of the plurality of conditions can be resulted in.

4. The artificial intelligence apparatus of claim 1, wherein the policy intelligence includes a treatment method planning intelligence for planning an appropriate treatment method through a prediction based on the condition of the patient, and a treatment history determination intelligence for determining the treatment history of the patient based on the condition of the patient and the predicted treatment method, and
wherein the global policy intelligence includes a global treatment method planning intelligence and a global treatment history determination intelligence.

5. The artificial intelligence apparatus of claim 1, wherein the global policy intelligence management module receives a federation message or a synchronization message from an external medical institution, or sends a federation message or a synchronization message to an external medical institution.

6. The artificial intelligence apparatus of claim 5, wherein the global policy intelligence management module:
receives a learning result of a policy intelligence of the external medical institution from the external medical institution and updates the global policy intelligence, in response to the federation message; and
provides the global policy intelligence to the external medical institution in response to the synchronization message.

7. A method of operating an artificial intelligence apparatus, the method comprising:
receiving an electronic medical record (EMR) of a patient and converting the received EMR into an episode including a condition of the patient, a treatment method applied to the patient, and a treatment history of the patient;
training a patient condition predictive intelligence for predicting a following condition of the patient after applying the treatment method to the patient;
performing reinforcement learning of a policy intelligence for exploring an optimized treatment path for the patient based on the episode;
updating a global policy intelligence for the reinforcement learning of the policy intelligence based on the policy intelligence; and
outputting the optimized treatment path for the patient using the policy intelligence.

8. The method of claim 7, wherein the converting of the received EMR into the episode includes:
reading the EMR associated with the patient from an EMR database and initializing the episode;
separating the EMR associated with the patient into an examination record table and a treatment record table, and arranging the examination record table and the treatment record table in chronological order;
generating a treatment method identifier applied to the patient based on the treatment record table;
generating a first condition identifier of the patient before applying the treatment method and a second condition identifier of the patient after applying the treatment method, based on the examination record table;
generating a treatment history identifier of the patient after applying the treatment method based on the examination record table; and
updating the episode based on the treatment method identifier, the first condition identifier, the second condition identifier, and the treatment history identifier.

9. The method of claim 7, wherein the patient condition predictive intelligence is a time series mixed probability distribution model that predicts a plurality of conditions that can be resulted in when the treatment method is applied to the patient and a probability that each of the plurality of conditions can be resulted in.

10. The method of claim 7, wherein the policy intelligence includes a treatment method planning intelligence for planning an appropriate treatment method through a prediction based on the condition of the patient, and a treatment history determination intelligence for determining the treatment history of the patient based on the condition of the patient and the predicted treatment method, and
wherein the global policy intelligence includes a global treatment method planning intelligence and a global treatment history determination intelligence.

11. The method of claim 10, wherein the performing of the reinforcement learning of the policy intelligence includes:
sampling the episode associated with the patient from an episode database;
synchronizing the treatment method planning intelligence and the treatment history determination intelligence with the global treatment method planning intelligence and the global treatment history determination intelligence;
adjusting a first weight for the treatment method planning intelligence and a second weight for the treatment history determination intelligence;
predicting a treatment method associated with the patient through the treatment method planning intelligence;
predicting the condition of the patient and the treatment history of the patient when the treatment method is applied to the patient through the patient condition prediction intelligence;
generating a first episode based on the treatment method, the condition, and the treatment history; and
updating parameters of the treatment method planning intelligence and the treatment history determination intelligence based on the first episode, and
wherein the first weight represents a ratio at which the parameters of the global treatment method planning intelligence are reflected in the treatment method planning intelligence, and the second weight represents a ratio at which the parameters of the global treatment history determination intelligence are reflected in the treatment history determination intelligence.

12. The method of claim 11, wherein the updating of the parameters includes:
sampling a second episode similar to the first episode from the episode database; and
updating the parameters of the treatment history determination intelligence based on the second episode.

13. The method of claim 7, wherein the updating of the global policy intelligence includes:
receiving a message from an external medical institution;
transmitting the global policy intelligence to the external medical institution when the message is a synchronization message; and
updating the global policy intelligence using a policy intelligence of the external medical institution provided from the external medical institution when the message is a federation message.

14. A non-transitory computer-readable medium comprising a program code that, when executed by a processor, causes the processor to execute operations of:
receiving an electronic medical record (EMR) of a patient and converting the received EMR into an episode including a condition of the patient, a treatment method applied to the patient, and a treatment history of the patient;
training a patient condition predictive intelligence for predicting a following condition of the patient after applying the treatment method to the patient;
performing reinforcement learning of a policy intelligence for exploring an optimized treatment path for the patient based on the episode;
outputting the optimized treatment path for the patient using the policy intelligence; and
updating a global policy intelligence for exploring the optimized treatment path based on the policy intelligence.

15. The non-transitory computer-readable medium of claim 14, wherein the converting of the received EMR into the episode includes:
reading the EMR associated with the patient from an EMR database and initializing the episode;
separating the EMR associated with the patient into an examination record table and a treatment record table, and arranging the examination record table and the treatment record table in chronological order;
generating a treatment method identifier applied to the patient based on the treatment record table;
generating a first condition identifier of the patient before applying the treatment method and a second condition identifier of the patient after applying the treatment method, based on the examination record table;
generating a treatment history identifier of the patient after applying the treatment method based on the examination record table; and
updating the episode based on the treatment method identifier, the first condition identifier, the second condition identifier, and the treatment history identifier.

16. The non-transitory computer-readable medium of claim 14, wherein the patient condition predictive intelligence is a time series mixed probability distribution model that predicts a plurality of conditions that can be resulted in when the treatment method is applied to the patient and a probability that each of the plurality of conditions can be resulted in.

17. The non-transitory computer-readable medium of claim 14, wherein the policy intelligence includes a treatment method planning intelligence for planning a treatment method through a prediction based on the condition of the patient, and a treatment history determination intelligence for determining the treatment history of the patient based on the condition of the patient and the predicted treatment method, and
wherein the global policy intelligence includes a global treatment method planning intelligence and a global treatment history determination intelligence.

18. The non-transitory computer-readable medium of claim 17, wherein the performing of the reinforcement learning of the policy intelligence includes:
sampling the episode associated with the patient from an episode database;
synchronizing the treatment method planning intelligence and the treatment history determination intelligence with the global treatment method planning intelligence and the global treatment history determination intelligence;
adjusting a first weight for the treatment method planning intelligence and a second weight for the treatment history determination intelligence;
predicting a treatment method associated with the patient through the treatment method planning intelligence;
predicting the condition of the patient and the treatment history of the patient when the treatment method is applied to the patient through the patient condition prediction intelligence;
generating a first episode based on the treatment method, the condition, and the treatment history; and
updating parameters of the treatment method planning intelligence and the treatment history determination intelligence based on the first episode, and
wherein the first weight represents a ratio at which the parameters of the global treatment method planning intelligence are reflected in the treatment method planning intelligence, and the second weight represents a ratio at which the parameters of the global treatment history determination intelligence are reflected in the treatment history determination intelligence.

19. The non-transitory computer-readable medium of claim 18, wherein the updating of the parameters includes:
sampling a second episode similar to the first episode from the episode database; and
updating the parameters of the treatment history determination intelligence based on the second episode.

20. The non-transitory computer-readable medium of claim 14, wherein the updating of the global policy intelligence includes:
receiving a message from an external medical institution;
transmitting the global policy intelligence to the external medical institution when the message is the synchronization message; and
updating the global policy intelligence using a policy intelligence of the external medical institution provided from the external medical institution when the message is a federation message.
